(19) United States
(12) Patent Application Publication
(10) Pub. No.: US 2004/0236343 A1
(43) Pub. Date: Nov. 25, 2004

(54) Insertion tool for ocular implant and method for using same

(76) Inventors: Jon B. Taylor, Groton, MA (US); Edwin G. Lee, Burlington, MA (US); Margaret B. Mulhern, Groton, MA (US); Timothy J. Morrill, Plaislow, NH (US); Dana M. Cote, Saugus, MA (US)

Correspondence Address: Alan W. Fiedler, Esq., Becton, Dickinson and Company, 1 Becton Drive, Franklin Lakes, NJ, 07417-1880, US

(21) Appl. No.: 10/443,752

(22) Filed: May 23, 2003

(51) Int. Cl. A61F 11/00

(52) U.S. Cl. 606/108

(57) ABSTRACT

An insertion tool containing an elastomeric band, ejection pin and cartridge assembly for holding a transcornea shunt during insertion procedures. The proximal flange of the shunt is gently held in a visible position within an adjustable opening in the elastomeric band at the distal end of the ejection pin. A slide mechanism is provided along the exterior surface of the insertion tool to drive the cartridge assembly which applies tension to the elastomeric band, increasing the opening diameter securing the shunt and allowing gentle release of the shunt when desired. Alternative embodiments of the insertion tool utilize severable fibers, a transparent split-mylar sheet or diaphragm, a cantilever fork, a collet assembly or an elastomeric grip collet to gently hold and release the shunt during insertion.

### FIELD OF THE INVENTION

The present invention relates to a device for use with ocular and non-ocular implants and more particularly, an insertion tool and method for the controlled insertion of a transcornea shunt through the cornea of an eye to relieve intraocular pressure. The embodiment of the present invention is applicable in both transcorneal and transscleral applications.

### BACKGROUND OF THE INVENTION

Glaucoma, caused by optic nerve cell degeneration, is the second leading cause of preventable blindness in the world today. A major symptom of glaucoma is a high interocular pressure, or “IOP”, which is caused by the trabecular meshwork failing to drain enough aqueous humor fluid from within the eye. Glaucoma therapy therefore has been directed at protecting the optic nerve and preserving visual function by attempting to lower IOP using various methods, such as through the use of drugs or surgery, including surgical methods such as trabeculectomy and the use of implants.

Trabeculectomy is a very invasive surgical procedure in which no device or implant is used. Typically, a surgical procedure is performed to puncture, or reshape, the trabecular meshwork, by surgically creating a channel opening the sinus venosus. Another surgical technique used involves the use of implants within the eye, such as stems or shunts, which are typically quite large and are implanted during a surgically invasive procedure. These implants work to relieve internal eye pressure by permitting aqueous humor fluid to flow from the interior chamber, through the sclera, and into a conjunctive bleb over the sclera. These procedures are very labor intensive for the surgeons and are often subject to failure due to scaring and cyst formations.

One solution to the problems encountered involves using a transcornea shunt as shown in place in FIG. 1. The transcornea shunt has been developed to reduce the intraocular pressure in the eye by shunting aqueous humor fluid from the interior chamber of the eye, through the cornea, to the terafilum. The transcornea shunt is the first device to drain aqueous humor fluid through the cornea, which makes surgical implantation of the device less invasive and allows for surgery to be quicker than with other surgical options. Additional details of this new shunt are described in International Patent Application No. PCT/US01/00350, entitled “Systems And Methods For Reducing Intraocular Pressure”, filed on Jan. 5, 2001 and published on Jul. 19, 2001 under the International Publication No. WO 01/50943, the entire content of which is incorporated herein by reference.

The transcornea shunt is very small, however, and difficult to handle and manipulate during insertion. The transcornea shunt 10 of FIG. 1 is constructed having a first flange at a proximal end to anchor the shunt on the outside surface of the cornea, and a second flange at a distal end to anchor the shunt on the inside surface of the cornea. A shaft extends between the first and second flanges, and can include a filter provided to prevent bacteria from infiltrating the eye through the shunt. The shaft and filter also serve to control the flow rate of the aqueous humor fluid from the interior chamber of the eye to the outside surface of the cornea.

The transcornea shunt is inserted, or implanted, in the cornea through a small incision, sufficiently large to allow the second flange to be manipulated through the cornea while securing the shunt in place once positioned. Currently available surgical devices, however, are not ideally suited for gently gripping the transcornea shunt while leaving a portion of the shunt exposed to allow the surgeon to see the distal end of the shunt during the insertion procedure. Because of the small size and construction of the shunt, it is difficult to pick up and manipulate the shunt using currently available surgical devices and forceps, which also, in many cases, completely hide the shunt during insertion.

Attempts to develop shunt implantation tools include insertion tools that house the shunt in a tubular tip, and insert the shunt through a pressing motion against the surface of the cornea. Such insertion tools include a stiff tube and a plunger assembly, wherein the shunt is held within the tubular section of the assembly at the tip of the tool. When the tool is pressed down against the eye, the plunger pushes the hidden shunt out of the tubular tip and into the cornea incision. However, in such a device, the shunt is hidden during insertion. Also, there is no method for holding the shunt in the tube of the insertion tool. If the shunt is dry, it will fall out easily, and when the shunt is wet, it is very easily damaged when a plunger is applied to the shunt surface to force the shunt from the tube.

As noted above, another difficulty encountered with such insertion tools includes visibility during shunt installation. Typically, the shunt is being inserted into a very small incision, commonly between 1.0 and 1.5 mm. The tip of the insertion tool where the shunt is held often blocks the visibility of the incision, the shunt, or both. Even where the tube and tube tip is very small, the visibility of the surgeon is compromised. Still another difficulty encountered with such insertion tools includes the creation of unwanted force applied to the eye during shunt installation. In such devices, when the plunger is pushed down, the force required to push the shunt from the tube and into the incision is also transmitted to the surface of the eye, increasing intraocular pressure. This typically results in a two-handed operation, as the surgeon must counteract the force resulting from the insertion by holding the insertion tip. Additionally, as visibility is impaired even further through the use of two hands, the risk of poor positioning can result, leading to the insertion tool not positioned properly and the shunt not entering the incision site. Additional details of such devices are discussed in U.S. patent application Ser. No. 60/175,658, referenced above.

Accordingly, a need exists for a tool for inserting a transcornea shunt through the cornea of the eye that can gently grasp, but also securely hold the proximal end of the shunt without damage to the delicate shunt structure, such that the incision and shunt are not hidden by the tool so that the surgeon can easily view, manipulate and insert the shunt through the cornea. Additionally, a need exists for a tool having the ability to continue to gently grasp the proximal end of the shunt after the distal end of the shunt has been inserted entirely through the cornea to provide user feedback regarding whether the distal flange has fully extended within the interior chamber of the eye. In addition, a need exists for a tool which can be fabricated to avoid introducing any additional damage to the delicate tissues of the eye.

### SUMMARY OF THE INVENTION

It is therefore an object of this invention to provide a device, and method for using the same, that may be used to insert a transcornea shunt through the cornea of the eye such that the shunt can be placed across the cornea to relieve IOP by draining the interior chamber of the eye of aqueous humor fluid.

Another object of the present invention is to provide a device for transcornea shunt installation which can gently, but also securely hold the proximal end of a shunt, such that the surgeon can easily view an incision and exposed shunt portion, manipulate and insert the shunt through the cornea.

Another object of the present invention is to provide a device for transcornea shunt installation which can gently hold the proximal end of the shunt after the distal end has been inserted through the cornea to provide user feedback regarding whether the distal flange has fully extended within the interior chamber of the eye.

Another object of the present invention is to provide a device for transcornea shunt installation which can be fabricated to avoid causing any damage to the delicate tissues of the eye

These and other objects are substantially achieved by providing a shunt insertion tool having a handle containing an elastomeric band and post assembly for gently holding a transcornea shunt during insertion procedures. The proximal flange of the shunt is held firmly within an adjustable opening in the elastomeric band at the distal end of the insertion tool handle. A slide mechanism is provided along the exterior surface of the insertion tool handle to drive an internal mechanism, applying tension to the elastomeric band and increasing the opening, allowing release of the shunt when desired.

### BRIEF DESCRIPTION OF THE DRAWINGS

The above and other objects and advantages will be apparent upon consideration of the following drawings and detailed description. The preferred embodiments of the present invention are illustrated in the appended drawings in which like reference numerals refer to like elements and in which:

FIG. 1 is an enlarged cross-sectional view a properly installed shunt, or ocular implant, that may be inserted using an insertion tool in accordance with an embodiment of the present embodiment;

FIG. 2 is an exploded perspective view of an embodiment of the present invention for properly inserting a shunt as shown in FIG. 1;

FIG. 3A is an exploded perspective view of a cartridge assembly for use in the embodiment of the present invention as shown in FIG. 2;

FIG. 3B is an enlarged perspective view of the distal end of a partially assembled cartridge assembly as shown in FIG. 3A;

FIG. 4 is a cross-sectional view of a cartridge assembly as shown in FIG. 3A;

FIG. 5A is a cross-sectional view of an embodiment of the present invention as shown in FIG. 2 in a insertion position;

FIG. 5B is a cross-sectional view of an embodiment of the present invention as shown in FIG. 2 in a release position;

FIG. 6 is an exploded perspective view of a second embodiment of the present invention for properly inserting a shunt as shown in FIG. 1;

FIG. 7A is a cross-sectional view of a second embodiment of the present invention as shown in FIG. 6 in a insertion position;

FIG. 7B is a cross-sectional view of a second embodiment of the present invention as shown in FIG. 6 in a release position;

FIG. 8A is an enlarged view of the distal end of a third embodiment of the present invention with a shunt attached and ready to install;

FIG. 8B is an enlarged view of the distal end of a third embodiment of the present invention with a shunt released from the insertion tool;

FIG. 9A is an enlarged view of the distal end of a fourth embodiment of the present invention with a shunt attached and ready to install;

FIG. 9B is an enlarged view of the distal end of a fourth embodiment of the present invention with a shunt released from the insertion tool;

FIG. 10A is an enlarged view of the distal end of a fifth embodiment of the present invention with a shunt attached and ready to install;

FIG. 10B is an enlarged view of the distal end of a fifth embodiment of the present invention with a shunt released from the insertion tool;

FIG. 11A is an enlarged view of the distal end of a sixth embodiment of the present invention with a shunt attached and ready to install;

FIG. 11B is an enlarged view of the distal end of a sixth embodiment of the present invention with a shunt released from the insertion tool;

FIG. 12A is an enlarged view of the distal end of a seventh embodiment of the present invention with a shunt attached and ready to install;

FIG. 12B is an enlarged view of the distal end of a seventh embodiment of the present invention with a shunt released from the insertion tool;

FIG. 13A is an enlarged view of the distal end of an eighth embodiment of the present invention with a shunt attached and ready to install;

FIG. 13B is an enlarged view of the distal end of an eighth embodiment of the present invention with a shunt released from the insertion tool; and

FIG. 13C is a cross-sectional view of a distal end of an eighth embodiment of the present invention as shown in FIG. 13A in a insertion position.

In the drawing figures, it will be understood that like numerals refer to like structures.

### DETAILED DESCRIPTION OF THE PREFERRED EMBODIMENTS

The transcornea shunt (hereinafter “shunt”) has been developed to reduce the intraocular pressure (IOP) in the eye by shunting aqueous humor fluid from the interior chamber of the eye, through the cornea, and to the terafilum. To do so, the shunt must be implanted through a small incision and into the cornea of the eye, actually extending between the inner and outer surface of the cornea. The shunt, however, is very small and difficult to handle and manipulate during such insertion procedures. In resolution of such problems, the embodiment of the present invention described below enables the surgeon to gently grasp the shunt at a distal end of a installation tool using a thin elastomeric material to hold the shunt in position until manually released. The elastomeric material can include any number of polymers, such as polybutadiene, polyisobutylene, and polyisoprene, or natural rubber. In the preferred, and each additional embodiment, only a small portion of the shunt is held during installation, allowing a large portion of the shunt to remain visible to the surgeon, allowing greater control and precision during installation.

The embodiments of the insertion tool described below include a substantially cylindrical housing, extending between distal and proximal ends with a shaft extending between each end. An elastomeric band is attached to extend over the distal end of the housing, and includes at least one opening for gently securing the shunt to the distal end of the housing. An ejector pin extends through the shaft of the housing, and is used to release the proximal end of the shunt from the elastomeric band during insertion.

At the distal end of the insertion tool housing, a small opening is provided in the elastomeric band having a diameter which can expand and contract relative to the degree of tension placed on the band. A flange at the proximal end of the shunt, or “head”, is gently held by the opening in the elastomeric band, such that when the band is relaxed, the head is gently, but securely held by the insertion tool. The shunt and elastomeric band are assembled onto an insertion tool which also includes a mechanism for applying, maintaining and releasing band tension during installation. The elastomeric band allows the shunt to be held such that the surgeon can manipulate the shunt into the cornea with complete control and maximum visibility of the insertion site. As only the head of the shunt is held, the flange at the distal end of the shunt, or “foot”, and shunt body remain visible to the surgeon during installation. When the surgeon has the shunt placed and positioned as desired, the surgeon can release the shunt by activating the mechanism for applying band tension by stretching the elastomeric band, such that the diameter of the opening located at the distal end of the insertion tool is enlarged and releases the shunt from its secured position.

As used herein, the term “proximal” refers to a location on any device closest to the person using the device and farthest from the patient in connection with which the device is used. Conversely, the term “distal” refers to a location on the device farthest from the person using the device and closest to the patient in connection with which the device is used.

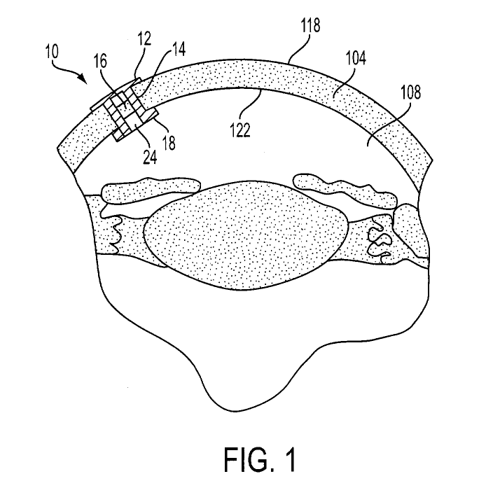

As shown in FIG. 1, the transcornea shunt is inserted through the cornea of an eye to relieve IOP by draining the interior chamber of the eye of aqueous humor fluid. FIG. 1 is an enlarged cross-sectional view of a properly installed transcornea shunt, or ocular implant, that may be inserted using an insertion tool in accordance with an embodiment of the present invention. The illustration of FIG. 1 shows a shunt 10 having a proximal flange, or head 12, a distal flange, or foot 18, and a body 14 extending between flanges, penetrating a cornea 104.

The cornea includes an inner and outer surface, 122 and 118 respectively, shielding an interior chamber 108 When properly installed, the head 12 contacts the outer corneal surface 118, and the foot 18 is located in the interior chamber 108 and contacts the inner corneal surface 122. The shunt 10 also includes a channel 24, passing through the body 14 between the foot 18 and the head 12, which is substantially open at the foot, and covered at the head. The cover of the channel 24 located at the head 12 includes a narrow opening, or slit (not shown), allowing fluid communication from within the interior chamber. The slit can also minimize ingress, control flow, and prevent bacteria infiltration. In yet another embodiment, the channel 24 can remain substantially open at the head 12, which allows an engagement with a centering mechanism on the insertion tool as described in greater detail below.

The shunt 10 can also include a replaceable filter 16 to prevent bacteria from infiltrating the eye through the shunt. The channel 24 and filter 16 also serve to control the flow rate of the aqueous humor fluid out of the interior chamber of the eye and to the outside surface of the cornea, providing a range of available flow rates. In yet another embodiment of the present invention, the filter 16 can be replaced or supplemented with a valve assembly, such as a one-way or check valve. In still another embodiment of the present invention, the narrow opening, or slit, located at the head 12 can serve as such a valve assembly. A filter 16 can be excluded in applications in which the shunt is to be used as an access port.

The shunt 10 can be constructed using a number of various materials, such as a shape memory polymer or a dehydrated hydrogel that swells in the incision when hydrated. The shunt 10 can also include different surface properties on different parts of the shunt. For example, one part can include a coating surface designed to promote cell adhesion. The shunt can also include a number of foot sizes, which correspond to a range of incision sizes depending upon the application, and can even allow slight dimensional alteration after implantation. Additional details of the shunt 10 are discussed in U.S. patent application Ser. No. 60/175,658, referenced above.

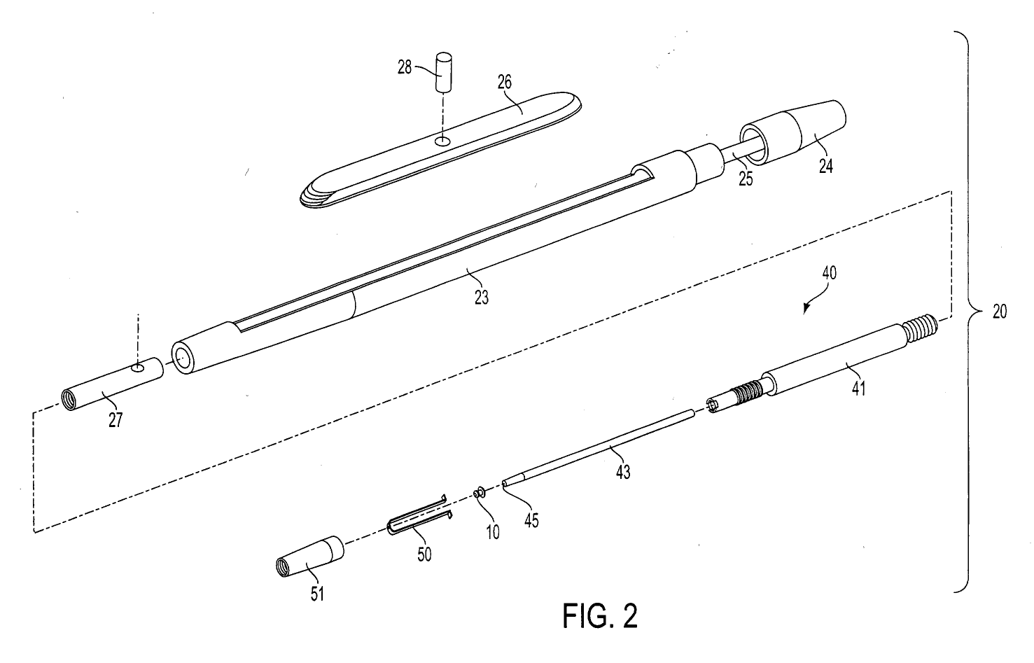

The embodiment of the present invention described below and shown in FIG. 2, illustrates an example of an insertion tool 20 that can be used to insert the shunt 10 shown in FIG. 1, through the corneal surface 104, such that the foot and head of the shunt are located in a correctly seated position. FIG. 2 is an exploded perspective view illustrating an example of an insertion tool 20 having a distal and proximal end, between which is located a cylindrical handle 23 defining a substantially hollow chamber in which a cartridge assembly 40 is located. The cylindrical handle 23 includes a first cap 24 mechanically attached to the proximal end, and an open distal end for purposes of containing, securing and inserting a transcornea shunt. A second cap (not shown) can be provided to mate with the distal end of the cylindrical handle 23 and to provide a secure, sterile barrier for the shunt held at the distal end. Additionally, a tamper indicator strip can be positioned over the seam formed between the second cap and the cylindrical handle 23. The strip can be perforated along the seam, and a ripped or torn perforation can indicate that the second cap and the cylindrical handle have been opened and that the shunt may no longer be sanitary.

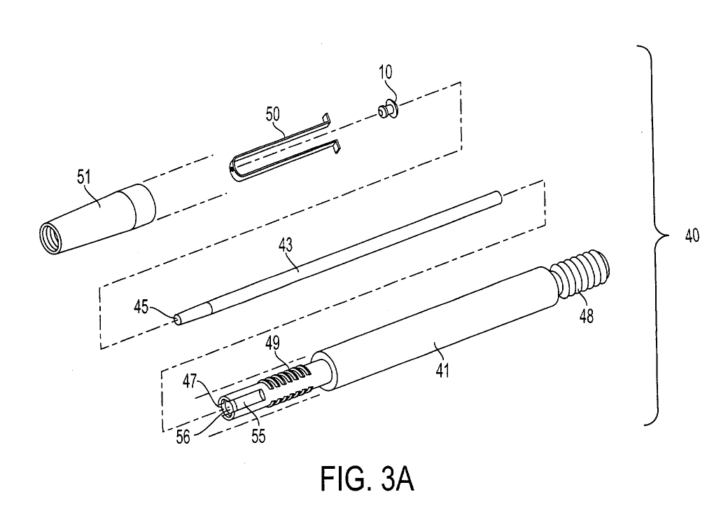

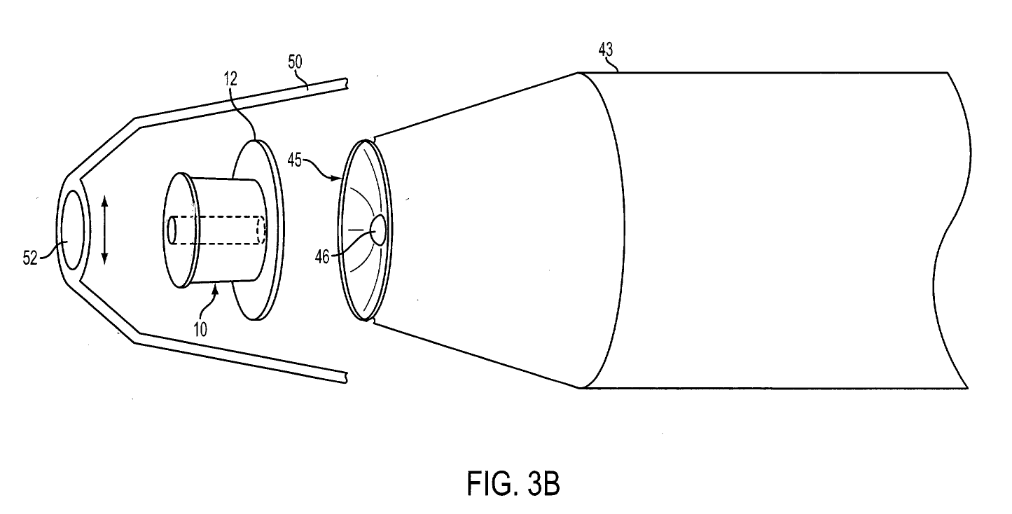

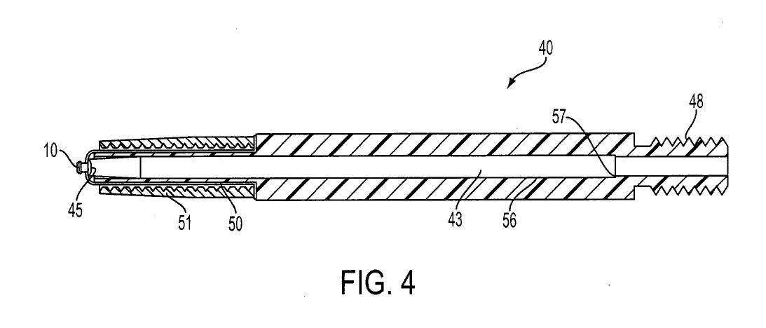

In FIG. 2, the first cap 24 includes a rod 25 extending from the cap into the handle 23 and into a cartridge assembly 40. An exploded perspective view of the cartridge assembly 40 is shown in FIG. 3A. The cartridge assembly of FIG. 3A includes a cartridge body 41, an ejection pin 43, an elastomeric band 50, and an outer sleeve 51. As shown in FIGS. 3A, 3B and 4, the cartridge body 41 is cylindrical about a substantially hollow chamber 56, and extends between distal and proximal ends, with the distal end having an attachment mechanism 49 for mechanically attaching the outer sleeve 51 to the cartridge body 41. The cartridge body 41 also includes a reduced outside diameter at the distal end such that when mechanically attached to the outer sleeve 51, a smooth, continuous surface is created to allow slidable movement within the handle 23. At the proximal end of the cartridge body 41, an attachment mechanism 48 is provided for mechanically attaching the cartridge body 41 to the slide block or cartridge connector 27 as discussed in greater detail below.

Referring again to FIG. 2, the cartridge body 41 of the cartridge assembly 40 is slidably contained with the substantially hollow handle 23 and is mechanically connected to the cartridge connector 27. The cartridge connector 27 is mechanically connected to the slide 26 via a pin assembly 28, thereby allowing the movement of the slide 26 to direct the movement of the cartridge body 41 within the substantially hollow handle 23. The cartridge body 41 is arranged to be slidably mounted within handle 23 for movement providing user control for applying and releasing tension to the elastomeric band 50 as described in greater detail below.

The attachment mechanisms 48 and 49 can be achieved through any number of suitable techniques, such as press-fit or mechanical threads as shown in FIGS. 3A and 4. The attachment mechanism 49 between cartridge body 41 and the outer sleeve 51 also works in cooperation with several additional features, described in greater detail below, located at the reduced diameter distal end of the cartridge body 41.

As shown in FIGS. 3A and 4, the cartridge body 41 includes two flat sides extending rearward along the outer surface of the cylindrical reduced diameter distal end, in alignment with a slot 47 provided at the distal end. Referring to FIG. 3A, the cartridge body 41 provides slot 47 between opposed flanges extending along the axis of the body at the distal end of the cartridge body 41. The slot and flanges provide a recessed circular opening at the distal end of the cartridge body 41 which is sized to accept the elastomeric band 50 and gently hold the proximal flange, or head of the shunt 10. The slot 47 also prevents the elastomeric band from sliding free of the distal end of the ejection pin 43 during use.

A substantially hollow shaft 56 extends through the cartridge body 41 from the proximal end to the distal end, and includes a shoulder 57 located near the proximal end of the body. The shaft 56 receives the ejection pin 43 through the distal end, with a distal end of the ejection pin 43 being tapered and having a concave tip 45 for supporting the head of the shunt 10 during insertion and release. The tip 45 of the ejection pin 43, shown in greater detail in FIG. 3B, can be concave to receive the head 12 of the shunt 10 which is gently held in place by an opening in the elastomeric band 50 which is extended across the concave tip 45. FIG. 3B is an enlarged perspective view of the distal end of a partially assembled cartridge assembly as shown in FIG. 3A. In FIG. 3B, the center of the concave tip 45 of the ejection pin 43 can include a centering mechanism 46, which mates with the channel opening located in the head 12 of the shunt 10. In doing so, the centering mechanism 46 aids in positioning the shunt and prevents unwanted axial shunt movement during installation. Where the shunt 10 does not provide a mateable channel opening, the centering mechanism 46 can be omitted.

When cartridge assembly 40 is assembled, the elastemeric band 50 is extended over distal end of the cartridge body 41, and fitted between the flanges within notch 47, and down each of the flat sides 55. The opening 52 in the elastomeric band 50 gently holds the head 12 of the shunt 10 within the concave portion 45 of the ejection pin 43 as shown in FIG. 3B. The ejection pin 43 is positioned in the shaft 56 and contacts the shoulder 57, thereby providing a firm support for the shunt 10 to be held in place by the elastomeric band 50, which is installed with a slight amount of tension sufficient to gently hold the shunt, but not of a degree where the opening 52 begins to significantly enlarge. As only the head of the shunt is held by the elastomeric band, the remaining shunt body remains fully visible to the surgeon during installation.

The elastomeric band 50, extended over the distal end of the ejection pin 43 and fitted between the flanges within notch 47, is attached to the cartridge body 41 along the flat sides 55 using adhesive, ultrasonic welding or any other bonding technique to retain the elastomeric band in position in a substantially low tension, or nearly relaxed condition. In this condition, the head of the shunt 10 is gently held by the opening 52 in the elastomeric band at the distal tip of the ejection pin 43. The elastomeric band 50 can also be held on the cartridge body 41 using the outer sleeve 51 to secure the ends and/or sides of the band when the sleeve is mechanically engaged with the cartridge body 41.

The elastomeric material of band 50 can include any number of polymers, such as polybutadiene, polyisobutylene, polyisoprene (natural rubber), poly(styrene-butadiene-acrylonitrile) or ABS, poly(styrene-acrylonitrile) or SAN, elastomeric polyolefins, polyamides or Nylon, Chloroprene rubber, silicone rubber, and polyurethanes. Still other elastomeric materials can include starches or sugars.

The selected thickness of such elastomeric material ranges from about 0.3 mil to 50 mil (0.0003 inch to 0.05 inch). The elastomeric material can be sterilized by steam, ETO, or irradiation. In band applications in which latex is used, the band functions at an optimal level after sterilization by steam. In the preferred embodiment, a polyurethane film with 3 mil thickness from Stevens Inc. (product code ST-625FS), sterilized by steam, showed an optimal elasticity level. Steam sterilization at 250 degrees Fahrenheit for no more than 30 minutes while the shunt and film are installed in the kit is a preferred sterilization technique of one embodiment of the present invention.

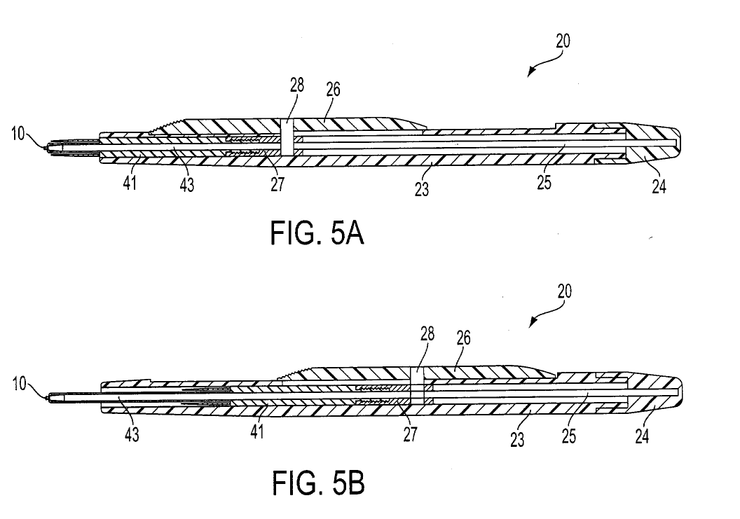

In FIGS. 5A and 5B, the insertion tool 20 is shown in use, releasing a shunt 10 after correctly positioning the shunt through a cornea. FIG. 5A is a cross-sectional view of an embodiment of the present invention in a insertion position, where the shunt is being gently held in a visible position for manipulation and insertion. FIG. 5B is a cross-sectional view of an embodiment of the present invention in a release position, where the shunt has been positioned, and is now released from the insertion device. In FIGS. 5A and 5B, the insertion tool 20 is shown gently holding the head of a shunt 10 in a visible position at the distal end of the ejection pin 43 using the elastomeric band 50 as described above. The shunt 10 is held such that the foot and body of the shunt 10 are visible and fully accessible for implantation by a surgeon. In this position, the shunt 10 can be properly implanted through the cornea such that the foot is contacting the inner corneal surface, while the head of the shunt 10 still being gently held by the insertion tool 20 using the elastomeric band 50. The holding of the shunt 10 after insertion allows the surgeon to obtain positive feedback on the correct implant-patient position by gently manipulating the insertion tool 20 and detecting resistance caused by the engagement between the foot of the shunt and the inner corneal surface. In addition, the surgeon can also rotate the insertion tool 20, which will also cause the shunt 10 to rotate, due to the rotation of the elastomeric band 50 and the ejection pin 43.

When the surgeon is satisfied with the implantation position, the secured ends of the elastomeric band 50 can then be retracted while the band 50 remains extended over the stationary ejection pin 43 as shown in FIG. 5B. In FIG. 5B, the slide 26, connected to cartridge connector 27, is manually slid rearward along the axis of the handle 23 by the surgeon holding the handle using one or more fingers. The cartridge body 41, mechanically engaged with the cartridge connector 27 and arranged to be slidably mounted within handle 23 for movement by the external slide 26, is therefore also slid rearward. The rod 25 engages the proximal end of the ejection pin 43 such that the ejection pin 43 remains stationary near the distal end of the handle 23 as the cartridge body 41 is retracted. As the cartridge body 41 is retracted rearward by the slide 26, tension in the elastomeric band 50 is increased as ejection pin 43 and rod 25 remain stationary and the secured ends of the elastomeric band 50 are retracted rearward with the cartridge body 41, creating an increased diameter in the opening 52 and thereby gently releasing the shunt 10. As shown in FIG. 5B, the cartridge body 41 has been fully retracted within the handle 23, and the elastomeric band 50 is under a maximum amount of tension, as it is extended across the distal end of the ejection pin 43 which remained stationary near the distal end of the handle 23. In this state, the opening 52 within the elastomeric band 50, formerly having a smaller diameter and gently holding the head of the shunt 10, has an increased diameter due to the applied tension, and in response, releases the shunt 10.

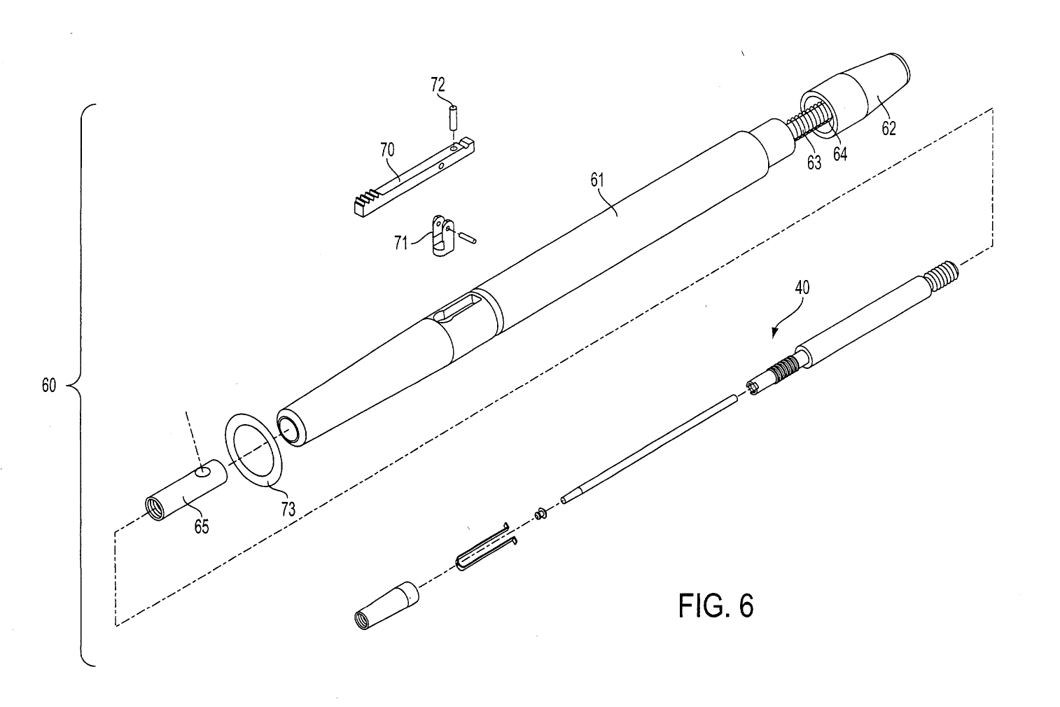

In a second embodiment of the present invention shown in FIG. 6, the cartridge assembly is substantially the same as described above. FIG. 6 is an exploded perspective view of a second embodiment of the present invention for properly inserting a shunt as shown in FIG. 1. In the insertion tool 60 of FIG. 6, the handle 61 of the second embodiment includes a first cap 62 on a proximal end, and a rod 63 extending from the cap into the handle 61. The first cap 62 is also attached to a spring 64 that extends into handle 61 and is connected to the cartridge connector 65 which includes an engagement mechanism for engaging the cartridge assembly, which is substantially the same as the cartridge assembly 40 in the first embodiment.

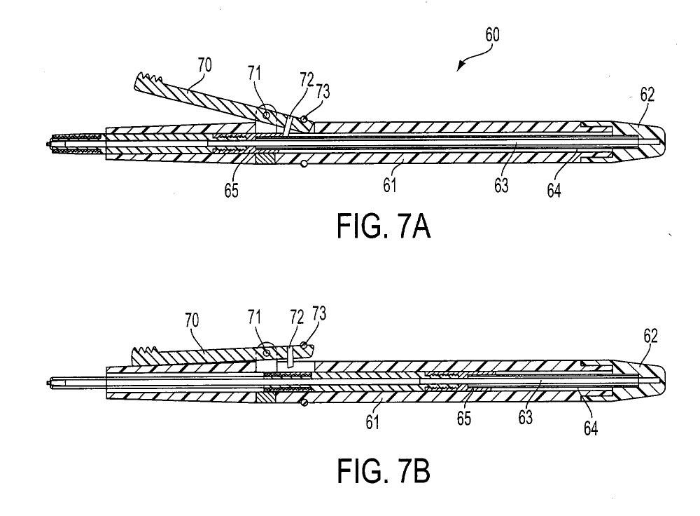

As shown in the cross-section view of FIGS. 7A and 7B, the cartridge connector 65 is held in position by a pin 72, and prevented from moving in the proximal direction. In this position, the spring 64 is applying tension on the cartridge connector 65, attempting to pull the connector rearward towards the proximal end of the handle 61. Handle 61 also includes a release mechanism 70 that pivots about pivot point 71 and includes pin 72 extending therefrom into handle 61 to retain the cartridge connector 65 in place. An elastic “O”-ring, or similar mechanism, is disposed in a groove located at the proximal end of the release mechanism 70, and keeps pin 72 engaged with the cartridge connector 65 until the release mechanism 70 is manually pivoted about point 71, lifting pin 72 from engagement with the cartridge connector 65, and allowing spring 64 to pull the connector rearward.

When pin 72 disengages from the cartridge connector 65, the cartridge connector 65 and cartridge body are retracted rearward by spring 64, creating tension in the elastomeric band as ejection pin 43 and rod 63 remain stationary and the secured ends of the elastomeric band 50 are retracted rearward with the cartridge body by spring 64, thereby releasing the shunt 10 substantially as described above. In this embodiment, the spring 64 provides the rearward motion required for shunt release.

In a modification of the embodiment shown in FIGS. 6, 7A and 7B, a damper mechanism can be added to prevent recoil during the rearward motion described above. The damper mechanism (not shown) can include for example, a small disc or washer installed between the connector 65 and spring 64 of FIG. 6. The disc includes a centered opening through which rod 63 extends, and has an opening diameter and contact surface sufficient to create friction between the disc and the rod 63 during rearward motion of the cartridge connector 65. The friction serves to slow the travel rate of the cartridge 40 and eliminates cartridge recoil. The disc can be fabricated from any suitable material, such as silicone.

In the embodiments of the present invention described above, the use of a concave tip at the distal end of the ejection pin 43 and an opening in an extended elastomeric band provide a very gentle grasp of the shunt body. The gentle grasp provided, or “soft interface” between shunt and insertion tool, prevents damage to the fragile shunt prior to, and during installation, and also provides greatly increased shunt visibility during installation.

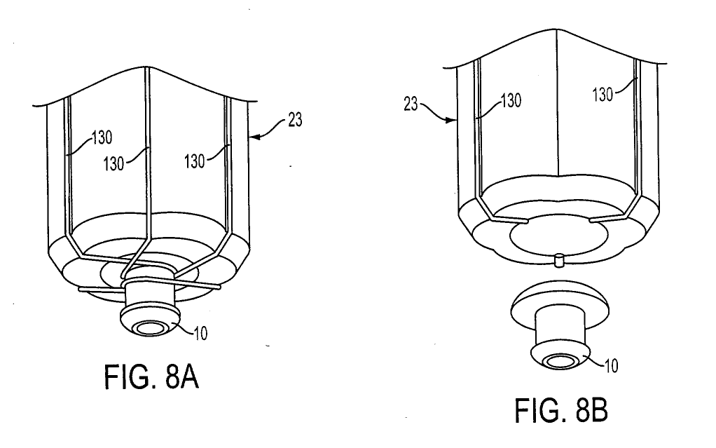

In yet another embodiment of the present invention shown in FIGS. 8A and 8B, a series of fibers can be used to hold the shunt 10 during insertion. FIG. 8A is an enlarged view of the distal end of a third embodiment of the present invention with a shunt held and ready to install. FIG. 8B is an enlarged view of the distal end of a third embodiment of the present invention with a shunt released from the insertion tool. The shunt 10 is gently held by the series of fibers 130 at the distal end of the insertion tool which is otherwise constructed substantially as described above. The diameter and composition of fibers can be varied to suit the specific application.

In FIGS. 8A and 8B, the shunt 10 is positioned at the distal end of the insertion device and held by a series of fibers 130 looped about the shunt 10, and terminated at a quick severing mechanism (not shown). When the cartridge body (not shown) is in a forward position, the series of fibers exert a force on the body of the shunt 10 as shown in FIG. 8A, gently holding the shunt at the distal end of the insertion device. When the cartridge body is retracted as described above, the quick severing mechanism is activated, severing the series of fibers 130 and releasing the shunt 10 to complete insertion.

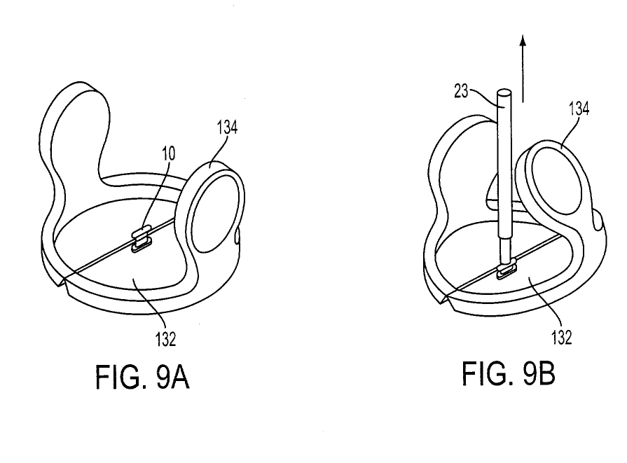

In yet another embodiment of the present invention shown in FIGS. 9A and 9B, a transparent split-mylar sheet having a perimeter frame and handles can be used to hold the shunt 10 during insertion. FIG. 9A is an enlarged view of the distal end of a fourth embodiment of the present invention with a shunt attached and ready to install. FIG. 9B is an enlarged view of the distal end of a fourth embodiment of the present invention with a shunt released from the insertion tool. The shunt 10 is gently held by the transparent, split-mylar diaphragm 132 at the distal end of the insertion tool which is otherwise constructed substantially as described above.

In FIGS. 9A and 9B, the shunt 10 is positioned at the distal end of the insertion device and held by opposing, semicircular planes of transparent mylar, creating a split-mylar diaphragm 132. When a perimeter frame and handles 134 are in a relaxed and closed position, the split-mylar diaphragm 132 is in a closed position and exerts a compressive force on the body of the shunt 10 as shown in FIG. 9A. The head of the shunt 10 is gently held within the split-mylar diaphragm 132 at the distal end of the insertion device. When the perimeter frame and handles 134 are retracted as shown in FIG. 9B, the split-mylar diaphragm 132 is separated, thereby releasing the shunt 10 completing insertion.

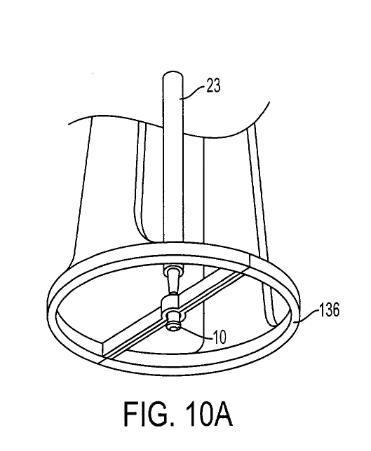

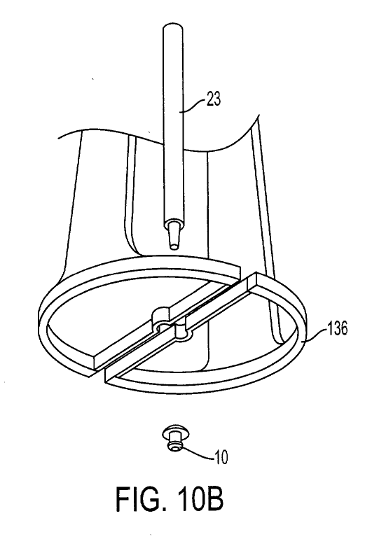

In yet another embodiment of the present invention shown in FIGS. 10A and 10B, a transparent, split-mylar diaphragm can be used without perimeter frame handles to hold the shunt 10 during insertion. FIG. 10A is an enlarged view of the distal end of a fifth embodiment of the present invention with a shunt attached and ready to install. FIG. 10B is an enlarged view of the distal end of a fifth embodiment of the present invention with a shunt released from the insertion tool. The shunt 10 is gently held by the transparent, split-mylar diaphragm 136 at the distal end of the insertion tool which is otherwise constructed substantially as described above.

In FIGS. 10A and 10B, the shunt 10 is positioned at the distal end of the insertion device and held by opposing semicircular frame members, creating a split-frame support 136. When the cartridge body (not shown) is in a forward position, the split-frame support 136 is in a closed position and exerts a compressive force on the body of the shunt 10 as shown in FIG. 10A. The head of the shunt 10 is gently held within the split-frame support 136 at the distal end of the insertion device. When the cartridge body (not shown) is retracted as described above, the split-frame support 136 is separated, thereby releasing the shunt 10 completing insertion as shown in FIG. 10B.

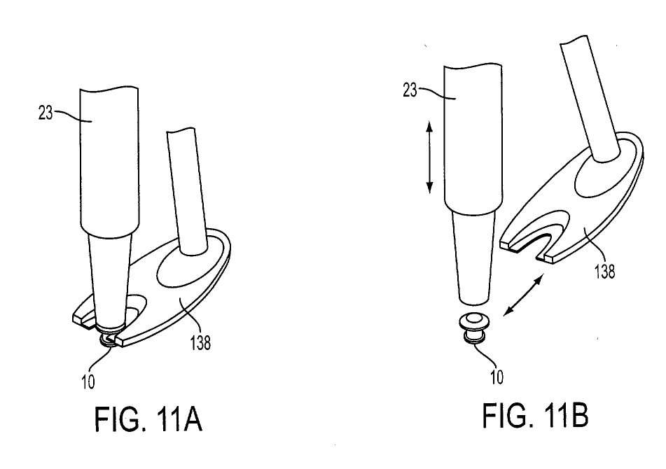

In yet another embodiment of the present invention shown in FIGS. 11A and 11B, a cantilever arm can be used to hold the shunt 10 during insertion. FIG. 11A is an enlarged view of the distal end of a sixth embodiment of the present invention with a shunt attached and ready to install. FIG. 11B is an enlarged view of the distal end of a sixth embodiment of the present invention with a shunt released from the insertion tool. The shunt 10 is gently held by the cantilever fork 138 at the distal end of the insertion tool which is otherwise constructed substantially as described above.

In FIGS. 11A and 11B, the shunt 10 is positioned at the distal end of the insertion device and held by a cantilever fork in an extended position. When the cartridge body (not shown) is in a forward position, the cantilever fork 138 is in an extended position and exerts a compressive force on the body of the shunt 10 as shown in FIGS. 11A and 11B. The head of the shunt 10 is gently held within the cantilever fork 138 at the distal end of the insertion device. When the cartridge body is retracted as described above, the cantilever fork 138 is retracted, thereby releasing the shunt 10 and completing insertion.

The perimeter frame and handles of FIG. 9, the split-frame of FIG. 10, and the cantilever of FIG. 11, can be constructed of transparent, or semi-transparent materials to maximize shunt visibility during installation. In embodiments which incorporate mylar, the mylar can be transparent, or slightly colored, to also assist in maximizing shunt visibility during installation.

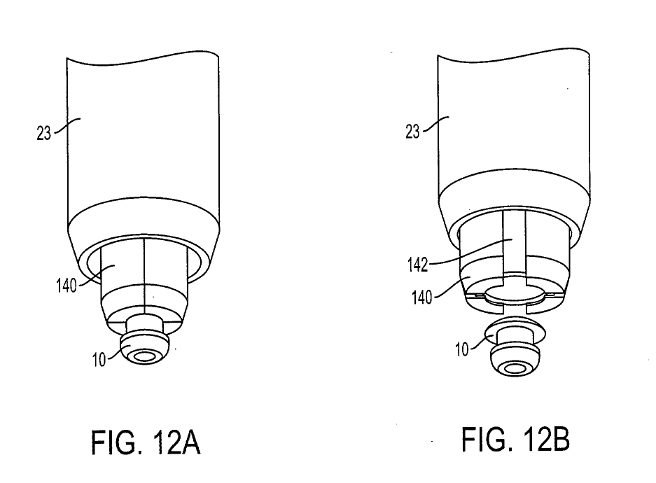

In yet another embodiment of the present invention shown in FIGS. 12A and 12B, a collet assembly can be used to hold the shunt 10 during insertion. FIG. 12A is an enlarged view of the distal end of a seventh embodiment of the present invention with a shunt attached and ready to install. FIG. 12B is an enlarged view of the distal end of a seventh embodiment of the present invention with a shunt released from the insertion tool. The shunt 10 is gently held within a pocket at the distal end of the insertion tool which is otherwise constructed substantially as described above.

In FIGS. 12A and 12B, the shunt 10 is positioned in a pocket created by a pad 142 located within a collet assembly 140 at the distal end of the insertion device. When the cartridge body (not shown) is in a forward position, the collet assembly exerts a compressive force on the body of the shunt 10 as shown in FIG. 12A. The head of the shunt 10 is gently held within the collet assembly 140, and is cushioned and stabilized by a pad 142. When the cartridge body is retracted as described above, the collet assembly 140 releases the shunt 10 to complete insertion.

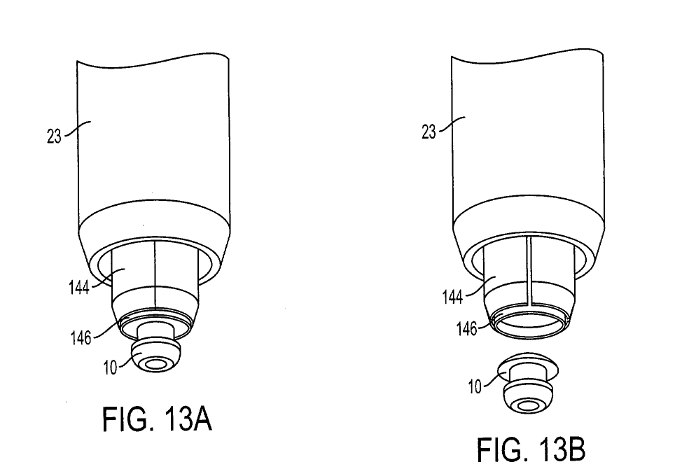

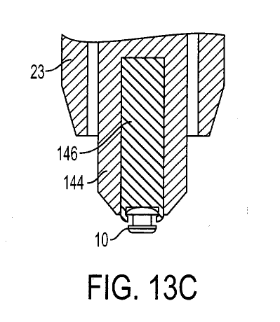

In yet another embodiment of the present invention shown in FIGS. 13A, 13B and 13C, an elastomeric grip collet can be used to hold the shunt 10 during insertion. FIG. 13A is an enlarged view of the distal end of an eighth embodiment of the present invention with a shunt attached and ready to install. FIG. 13B is an enlarged view of the distal end of an eighth embodiment of the present invention with a shunt released from the insertion tool, and FIG. 13C is a cross-sectional view of a distal end of an eighth embodiment of the present invention as shown in FIG. 13A in a insertion position. The shunt 10 is gently held within an elastomeric pocket 146 at the distal end of the insertion tool which is otherwise) constructed substantially as described above.

In FIGS. 13A, 13B and 13C, the shunt 10 is positioned in an elastomeric pocket 146, which is positioned within a collet assembly 144 at the distal end of the insertion device. When the cartridge body (not shown) is in a forward position, the collet assembly exerts a compressive force on the elastomeric pocket 146 as shown in FIGS. 13A and 13C. The pliable elastomeric pocket responds to this applied force by disposing slightly about the head of the shunt 10, thereby gently holding the shunt. When the cartridge body is retracted as described above, the collet assembly 144 no longer compresses the elastomeric pocket 146 and the shunt 10 is released.

The insertion tool embodiments described above each have the advantage of allowing the surgeon to manipulate the shunt in the implantation site in the cornea before it is actually released from the insertion tool, allowing the surgeon to remain confident that the shunt is correctly positioned. Also, a minimal amount of force is actually transmitted to the eye from the insertion tool and insertion process, allowing one handed operation during insertion. In doing so, the tool and method described above requires much less skill for shunt implantation procedures.

In the embodiments described above, the shunt remains highly visible during installation due to the fact the shunt is fully exposed at the end of the device. The shunt is prevented from falling from the device prior to installation, and such attachment allows the surgeon an increased level of control during installation. For example, the surgeon has the flexibility to put one side of the shunt in first, and then twist and roll the shunt in order to complete implantation in the small incision. Although the embodiments are described in use with a shunt, any similar device can also be installed using the systems and methods described above. These similar devices can include transcornea drug delivery devices, which are constructed similarly to the shunt described above. Additionally, the embodiments of the present invention described above can be used in both transcorneal applications as described above, or in transscleral applications.

Although only a few exemplary embodiments of the present invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims.

What is claimed is:

1. An insertion tool for use with implants for ophthalmic and non-ophthalmic applications, comprising:
a handle body having a distal end, a proximal end, and a substantially cylindrical housing extending along a longitudinal axis between said distal and proximal ends of said handle body, said cylindrical housing defining a chamber therein and having at least one opening accessing said chamber, said chamber open at said distal end of said handle body;
a movable cartridge disposed in said chamber for longitudinal movement between a shunt securing position and a shunt releasing position with respect to said handle body, said cartridge having a proximal end and a distal end, said distal end of said cartridge securing a shunt when said cartridge is in said shunt securing position and releasing said shunt when said cartridge is in said shunt releasing position; and
a cartridge positioning mechanism slidably mounted along an outer surface of said handle body and engaged with said cartridge via said opening accessing said chamber for moving said cartridge from between said shunt securing position and said shunt releasing position.

2. An insertion tool for use with implants for ophthalmic and non-ophthalmic applications as claimed in claim 1, wherein:
said proximal end of said handle body includes a rod extending along a longitudinal axis into said chamber.

3. An insertion tool for use with implants for ophthalmic and non-ophthalmic applications as claimed in claim 2, wherein said movable cartridge comprises:
a cartridge body having a substantially cylindrical housing extending along a longitudinal axis between said distal and proximal ends of said cartridge;
said proximal end, having a first engagement mechanism for engaging said cartridge positioning mechanism, and having an access port allowing said rod to access said chamber;
said distal end, having a second engagement mechanism for engaging an outer sleeve;
an ejection pin, having a distal and proximal end, said access port allowing said rod to maintain said pin in a stationary position as said cartridge is moved; and
an elastomeric band, having at least one opening of an adjustable diameter and being disposed over said distal end of said ejection pin, said opening securing said shunt against said distal end of said ejection pin when said cartridge is in said shunt securing position and releasing said shunt when said cartridge is in said shunt releasing position.

4. An insertion tool for use with implants for ophthalmic and non-ophthalmic applications as claimed in claim 3, wherein:
said elastomeric band includes a first and second end, said first and second end secured to said cartridge body, wherein said longitudinal movement of said cartridge body between said shunt securing position and said shunt releasing position varies a tension level in said elastomeric band and controls said adjustable diameter of said opening.

5. An insertion tool for use with implants for ophthalmic and non-ophthalmic applications as claimed in claim 3, wherein:
said adjustable diameter of said opening is adjustable from between a first and second diameter based upon a tension level in said band, said first diameter sufficient to secure said shunt, and said second diameter sufficient to release said shunt.

6. An insertion tool for use with implants for ophthalmic and non-ophthalmic applications as claimed in claim 3, wherein:
said distal end of said ejection pin is concave to receive said shunt.

7. An insertion tool for use with implants for ophthalmic and non-ophthalmic applications as claimed in claim 3, wherein said securing said shunt comprises:
locating a shunt proximal flange between said distal end of said ejection pin and said elastomeric band;
locating a shunt distal flange and shunt body within said opening of an adjustable diameter, said shunt distal flange and shunt body extending through said opening and beyond said elastomeric band, said locating exposing said shunt distal flange and shunt body during installation; and
locating said cartridge body in said shunt securing position, said locating creating a nearly relaxed tension in said elastomeric band and providing a first diameter sufficient to secure said shunt proximal flange against said distal end of said ejection pin.

8. An insertion tool for use with implants for ophthalmic and non-ophthalmic applications as claimed in claim 3, wherein said releasing said shunt comprises:
locating said cartridge body in said shunt releasing position, said locating creating a nearly maximum tension in said elastomeric band and providing a second diameter sufficient to release a shunt proximal flange from said distal end of said ejection pin.

9. An insertion tool for use with implants for ophthalmic and non-ophthalmic applications as claimed in claim 3, wherein:
said distal end of said ejection pin includes a centering mechanism to receive said shunt, said centering mechanism comprised of a raised surface disposed on said distal end of said ejection pin.

10. An insertion tool for use with implants for ophthalmic and non-ophthalmic applications as claimed in claim 3, wherein:
said cartridge body, ejection pin and elastomeric band comprise a soft interface for gently securing said shunt prior to and during installation and release.

11. An insertion tool for use with implants for ophthalmic and non-ophthalmic applications as claimed in claim 10, wherein:
said soft interface prevents damage to said shunt prior to and during installation and release, and provides maximum shunt visibility during installation and release.

12. An insertion tool for use with implants for ophthalmic and non-ophthalmic applications as claimed in claim 3, wherein:
at least one of said first and second engagement mechanism comprises a threaded fitting.

13. An insertion tool for use with implants for ophthalmic and non-ophthalmic applications as claimed in claim 1, wherein:
said moving said cartridge from between said shunt securing position and said shunt releasing position is mechanically assisted.

14. An insertion tool for use with implants for ophthalmic and non-ophthalmic applications as claimed in claim 13, wherein:
said mechanical assistance is a spring.

15. An insertion tool for use with implants for ophthalmic and non-ophthalmic applications as claimed in claim 13, wherein:
said mechanical assistance includes a damper mechanism.

16. An insertion tool for use with implants for ophthalmic and non-ophthalmic applications as claimed in claim 1, wherein:
said cartridge positioning mechanism comprises a user activated release engaged with said cartridge via said opening accessing said chamber.

17. An insertion tool for use with implants for ophthalmic and non-ophthalmic applications as claimed in claim 1, wherein:
said movable cartridge and said cartridge positioning mechanism are adapted to release a plurality of fibers, said fibers securing said shunt.

18. An insertion tool for use with implants for ophthalmic and non-ophthalmic applications as claimed in claim 1, wherein:
said movable cartridge and said cartridge positioning mechanism are adapted to release a split mylar sheet, said split mylar sheet securing said shunt.

19. An insertion tool for use with implants for ophthalmic and non-ophthalmic applications as claimed in claim 18, wherein said split mylar sheet is of at least one of a transparent, semi-transparent and colored material.

20. An insertion tool for use with implants for ophthalmic and non-ophthalmic applications as claimed in claim 1, wherein:
said movable cartridge and said cartridge positioning mechanism are adapted to release a split frame, said split frame securing said shunt.

21. An insertion tool for use with implants for ophthalmic and non-ophthalmic applications as claimed in claim 20, wherein said split frame is of at least one of a transparent, semi-transparent and colored material.

22. An insertion tool for use with implants for ophthalmic and non-ophthalmic applications as claimed in claim 1, wherein:
said movable cartridge and said cartridge positioning mechanism are adapted to release a cantilever, said cantilever securing said shunt.

23. An insertion tool for use with implants for ophthalmic and non-ophthalmic applications as claimed in claim 22, wherein said cantilever is at least one of a transparent, semi-transparent and colored material.

24. An insertion tool for use with implants for ophthalmic and non-ophthalmic applications as claimed in claim 1, wherein:
said movable cartridge and said cartridge positioning mechanism are adapted to release a collet jaw, said collet jaw securing said shunt.

25. An insertion tool for use with implants for ophthalmic and non-ophthalmic applications as claimed in claim 1, wherein said insertion tool is used in transcorneal applications.

26. An insertion tool for use with implants for ophthalmic and non-ophthalmic applications as claimed in claim 1, wherein said insertion tool is used in transscleral applications.

27. An insertion cartridge for use with implants for ophthalmic and non-ophthalmic applications, wherein said cartridge comprises:
a cartridge body having a substantially cylindrical housing extending along a longitudinal axis between said distal and proximal ends of said cartridge;
an ejection pin, disposed within said cartridge body and having a distal and proximal end, said ejection pin remaining in a stationary position during longitudinal movement of said cartridge body; and
an elastomeric band, having at least one opening of an adjustable diameter and being disposed over said distal end of said ejection pin, said opening securing a shunt against said distal end of said ejection pin when said cartridge body is in said shunt securing position and releasing said shunt when said cartridge body is in said shunt releasing position.

28. An insertion cartridge for use with implants for ophthalmic and non-ophthalmic applications as claimed in claim 27, wherein:
said elastomeric band includes a first and second end, said first and second end secured to said cartridge body, wherein said longitudinal movement of said cartridge body between said shunt securing position and said shunt releasing position varies a tension level in said elastomeric band and controls said adjustable diameter of said opening.

29. An insertion cartridge for use with implants for ophthalmic and non-ophthalmic applications as claimed in claim 27, wherein:
said adjustable diameter of said opening is adjustable from between a first and second diameter based upon a tension level in said band, said first diameter sufficient to secure said shunt, and said second diameter sufficient to release said shunt.

30. An insertion cartridge for use with implants for ophthalmic and non-ophthalmic applications as claimed in claim 27, wherein:
said distal end of said ejection pin is concave to receive said shunt.

31. An insertion cartridge for use with implants for ophthalmic and non-ophthalmic applications as claimed in claim 27, wherein:
said distal end of said ejection pin includes a centering mechanism to receive said shunt, said centering mechanism comprised of a raised surface disposed on said distal end of said ejection pin.

32. An insertion cartridge for use with implants for ophthalmic and non-ophthalmic applications as claimed in claim 27, wherein said securing said shunt against said distal end of said ejection pin comprises:
locating a shunt proximal flange between said distal end of said ejection pin and said elastomeric band;
locating a shunt distal flange and shunt body within said opening of an adjustable diameter, said shunt distal flange and shunt body extending through said opening and beyond said elastomeric band, said locating exposing said shunt distal flange and shunt body during installation; and
locating said cartridge body in said shunt securing position, said locating creating a nearly relaxed tension in said elastomeric band and providing a first diameter sufficient to secure said shunt proximal flange against said distal end of said ejection pin.

33. An insertion cartridge for use with implants for ophthalmic and non-ophthalmic applications as claimed in claim 27, wherein said releasing said shunt comprises:
locating said cartridge body in said shunt releasing position, said locating creating a nearly maximum tension in said elastomeric band and providing a second diameter sufficient to release a shunt proximal flange from said distal end of said ejection pin.

34. An insertion cartridge for use with implants for ophthalmic and non-ophthalmic applications as claimed in claim 27, wherein:
said cartridge body, ejection pin and elastomeric band comprise a soft interface for gently securing said shunt prior to and during installation and release.

35. An insertion cartridge for use with implants for ophthalmic and non-ophthalmic applications as claimed in claim 34, wherein:
said soft interface prevents damage to said shunt prior to and during installation and release, and provides maximum shunt visibility during installation and release.

36. A method for releasably securing and installing an implant in ophthalmic and non-ophthalmic applications, the method comprising:
releasably securing a proximal portion of an implant to a handle body having a distal end, a proximal end, and a substantially cylindrical portion extending along a longitudinal axis between said distal and proximal ends of said handle body, said releasably securing of said proximal portion of said implant leaving said distal portion of said implant visible to a user;
positioning said implant within an implant location, said positioning effectively disposing said distal portion of said implant within said implant location and said releasable securing of said proximal portion of said implant allowing control of said implant during and after said positioning of said implant within said implant location; and
releasing said proximal portion of said implant.

37. A method for releasably securing and installing an implant in ophthalmic and non-ophthalmic applications as claimed in claim 36, wherein said releasably securing a proximal portion of an implant comprises a soft interface between said implant and said handle body.

38. A method for releasably securing and installing an implant in ophthalmic and non-ophthalmic applications as claimed in claim 37, wherein said soft interface includes at least one of an elastomeric band having a variable opening for receiving and securing said implant, and an ejection pin having a concave end for positioning said implant.
